(19) United States
(12) Patent Application Publication
(10) Pub. No.: US 2021/0386141 A1
(43) Pub. Date: Dec. 16, 2021

(54) Transparent flexible self-attaching face mask

(71) Applicant: Alice M. Chun, New York, NY (US)

(72) Inventor: Alice M. Chun, New York, NY (US)

(21) Appl. No.: 17/321,547

(22) Filed: May 17, 2021

Related U.S. Application Data
(60) Provisional application No. 63/038,744, filed on Jun. 12, 2020.

(51) Int. Cl. A41D 13/11; A62B 18/08; A62B 23/02; A62B 18/10

(52) U.S. Cl. CPC A41D 13/1176; A62B 18/084; A62B 18/08; A62B 18/10; A62B 23/02

(57) ABSTRACT

A reusable transparent flexible silicone mask has a border coated with hydrogel or non-toxic emulsion adhesive that adheres directly to a wearer's face by hydrogel emulsion adhesion, without ear elastics or ties around the back of the head. The mask protects against breathing pathogens. The mask has a half-face version that covers the wearer's nose and mouth, a goggles version, and a full-face version. The mask may include one or more of a removable air filter cartridge, a breathing valve, a headlamp, a glasses gate, and a headband for keeping the mask on the wearer's body even when the mask is not affixed to the face.

### CROSS-REFERENCE TO RELATED APPLICATIONS

This application claims priority from U.S. patent application 63/038,744, filed Jun. 12, 2020, having a common inventor herewith; and this priority application is hereby incorporated by reference in its entirety in this application.

### BACKGROUND OF THE INVENTION

The present invention relates to a face mask for wearing by a human, and more particularly, is directed to a reusable transparent flexible face mask that adheres to the wearer's face and has air filters for protection against breathing pathogens.

A face mask function to protect its wearer from airborne undesirable substances, and sometimes to protect the environment from undesirable exhalations of the wearer. Because faces play an important role in socializing and communication, masks designed for pathogen protection often have unintended and undesirable social consequences.

U.S. Pat. No. 3,438,370 (Krantz) shows a face mask with ties that fasten behind a user's head, formed of a thin transparent (clear) air-impervious rigid material such as plastic that can deform under pressure to the contour of the wearer's face, especially her nose. The mask has a flap valve positioned under the user's nose and above her mouth, that opens during inhalation. The lower portion of the mask has a passageway for discharging mouth exhalations towards the wearer's neck, the passageway filled with granular material, such as activated coconut charcoal, for absorbing bacteria and viruses, thus purifying exhaled air.

The Krantz mask does not protect its wearer from inhaling ambient bacteria and viruses, it discourages spread of bacteria and viruses exhaled by the wearer.

U.S. Pat. No. 10,524,525 (Yazdi) discloses a suit that protects healthcare workers from air-born infection. The suit includes a hood having a clear plastic front portion extending over a wearer's face providing 360° protection, the hood having a cowl extending over the shoulders of the wearer. The front portion has an integrated respirator located over the wearer's nose. The back of the headgear has vents to reduce fogging, and sweat absorbent pads inside the visor. Another version of the hood has inhalation vents and an isolated exhalation pathway. The clear face shield increases the wearer's field of vision, and improves patient perception of health care workers.

People going about their daily business would prefer to not wear safety hoods as they are cumbersome and can impede sound transmission.

U.S. Pat. No. 10,834,978 (Yelken) discloses an opaque face mask edged with a soft silicone cushion to protect a user from facial skin damage or discomfort due to ties or elastic straps around the ears or back of head exerting pressure on the face, and to provide a seal against the user's face for preventing exhaled air leakage at the lateral edges of the face mask. One version of the mask has a nose grid for filtering inhaled and exhaled air. Another version of the mask may be made of cloth that filters inhaled and exhaled air. Most existing face masks may be retrofitted with an edged silicone cushion.

Deaf people usually rely on facial speech reading and facial cues, that are blocked by the Yelken mask. Masks to help with lip reading feature a clear plastic rectangle inset into a standard surgical mask or cloth face covering, but they often fog, and they cover the nose and cheeks with opaque material. Less than half of spoken English is visible on the lips alone, and speech-readers often rely on context clues from the speaker's face holistically, so any covering is an impediment to communication.

Some people have expressed fear of being mistaken for an assailant, and consequent police violence, if they wear a mask.

There is room for an improved face mask.

### SUMMARY OF THE INVENTION

In accordance with an aspect of this invention, there is provided a face mask for a human wearer, comprising a shield and a hydrogel emulsion adhesion layer. The shield is for covering a portion of a face of the wearer, the shield being formed of a material that blocks airborne pathogens, the shield having a border area on one side of the shield that extends around a perimeter of the shield. The hydrogel layer covers the border area of the shield, and is for removably adhering to the wearer's face via hydrogel adhesion, so that the face mask can be worn without a structure for applying force to keep the face mask on the wearer's face.

The material of the shield of the face mask may be clear flexible silicone.

The face mask may cover (a) a lower portion of the wearer's face including the wearer's mouth and nostril openings, (h) an upper portion of the wearer's face including the wearer's eyes, or (c) a majority of the wearer's face.

The face mask may include one or more of (1) at least one removable air filter cartridge that inserts into and removes from the shield, (2) a breathing valve that opens during exhalation and closes during inhalation, (3) a headlamp for illuminating what the wearer is looking at, (4) a glasses gate for enabling arms of glasses worn by the wearer to extend through the mask and over the wearer's ears, and (5) a headband for keeping the mask on the wearer's body even when the mask is not affixed to the wearer's face by the hydrogel layer.

### BRIEF DESCRIPTION OF THE DRAWINGS

FIG. 1 is a frontal view of half-mask 100A being worn;

FIG. 2 is a frontal view of half-mask 100A when not worn;

FIG. 3A is a profile view of half-mask 100A being worn;

FIG. 3B is a profile view of half-mask 100B being worn;

FIG. 3C shows headband 180 of half-mask 100B;

FIGS. 4A-4D are views of the side-of-face air filter cartridge used in half-mask 100A;

FIG. 4E is a view of the bottom-of-face air filter cartridge used in half-mask 100A;

FIGS. 5A-5B are views of the side-of-face air filter cartridge used in half-mask 100C;

FIGS. 6A-6C are views of the breathing valve used in half-mask 100A;

FIG. 7A is a frontal view of mask 200A being worn;

FIG. 7B is a frontal view of mask 200A when not worn;

FIG. 8 is a profile view of mask 200A being worn;

FIGS. 9A-9C are views of glasses gate 240 of mask 200A;

FIG. 9D is a frontal view of mask 200B when not worn;

FIGS. 9E-9F are views of glasses gate 440 of mask 200B;

FIGS. 10A-10C are views of headlamp 250;

FIG. 11 is a frontal view of goggles 300A and half-mask 100D being worn;

FIG. 12 is a profile view of goggles 300A and half-mask 100D being worn; and

FIG. 13 is a frontal view of goggles 300B and half-mask 100D when not worn.

### DETAILED DESCRIPTION

The present invention is a reusable transparent flexible face mask that adheres directly to a wearer's face, without ear elastics or ties around the back of the head. The face mask protects against breathing pathogens such as COVID-19, SARS, and other airborne viruses. The face mask is discussed below with respect to a half-face version that covers the bottom half of a wearer's face, a half-face version that covers the top half of a wearer's face (goggles) and a full-face version that covers the majority of a wearer's face from forehead to chin.

An adhesive border, based on scar healing tape used for burn victims, around the edge of the face mask, promotes skin health and healing, and eliminates dermatitis. Conventional masks create mechanical pressure to hold the mask to the wearer's face, causing dermatitis. The present mask uses hydrogel attraction to hold the mask to the wearer's face without ear loops or head ties.

The present mask uses removable air filter cartridges comprising an air filter and an air filter holder. Types of air filter cartridges include but are not limited to: (a) a washable air filter, that should be washed after about 10-15 hours of use, and can withstand at least 100 washings; (b) an air filter made of HEPA-like material typically used in vacuum cleaners and air purifiers; (c) an air filter made of conventional N95 material; and (d) non-conventional filter material using natural materials including a composite filter material of PLA melt blown fiber, bamboo, silk and/or cotton.

Because the face shield of the present mask is entirely clear, it permits facial communication such as lip-reading, smiles and other expressions, and the wearer does not look like an assailant. Because of its air-tight seal to the wearer's face, eyeglasses do not fog. The adhesive is sufficiently sticky for sweaty wearers such as athletes, when used in a humid environment, and for facial hair.

The goggles version of the present mask may include one or more of:
a removable air filter cartridge for reducing moisture and heat from the inside of the mask,
a headlamp for illuminating what the mask wearer is looking at,
a glasses gate for accommodating glasses worn by the wearer under the mask, that is, enabling the arms of the glasses to extend over the wearer's ears as usual, and
a headband, for keeping the mask on the wearer's body even when the mask is not affixed to the face.

The half-face mask for the lower half of the wearer's face may include one or more of:
at least one removable air filter cartridge for reducing moisture and heat from the inside of the mask, and for enabling the wearer to safely inhale and exhale in an environment containing airborne pathogens,
a breathing valve that opens during exhalation and closes during inhalation, and
a headband, for keeping the mask on the wearer's body even when the mask is not affixed to the face.

The full-face version of the present mask may include one or more of:
a removable air filter cartridge for reducing moisture and heat from the inside of the mask, and for enabling the wearer to safely inhale and exhale in an environment containing airborne pathogens,
a breathing valve that opens during exhalation and closes during inhalation,
a headlamp for illuminating what the mask wearer is looking at,
a glasses gate for accommodating glasses worn by the wearer under the mask, that is, enabling the arms of the glasses to extend over the wearer's ears as usual, and
a headband, for keeping the mask on the wearer's body even when the mask is not affixed to the face.

Disposal of the present mask is environmentally friendly. Conventional N95 mask filters release toxins during degradation, particularly harming the oceans. Billions of conventional masks are added to landfills annually. The present N95 filters are formed from polylactic acid (PLA)—a polymer derived from corn that the FDA has deemed safe for use in eating utensils-bamboo, cotton and silk; and these filters do not release toxins when they degrade. The present mask is formed of silicone, proven to be sturdy and durable from its use in cookware, and is easily cleaned and reused, reducing new landfill.

The present mask is simple to use, increasing the likelihood that it will be used, and relatively low cost.

The present face mask is useful for service providers who interact with patients or customers, such as police, medical personnel, and for the general public.

### Half-Face Mask

Mask 100 is a half-face mask that covers the bottom half of a wearer's face, having embodiments 100A, 100B and 100C. Mask 100A has silicone shield 110A, air filter cartridges 36 and breathing valve 133. Mask 100B comprises mask 100A with optional removable headband 180. Mask 100C has a different air filter cartridges than mask 100A and a different way of connecting the air filter cartridges to the silicone shield.

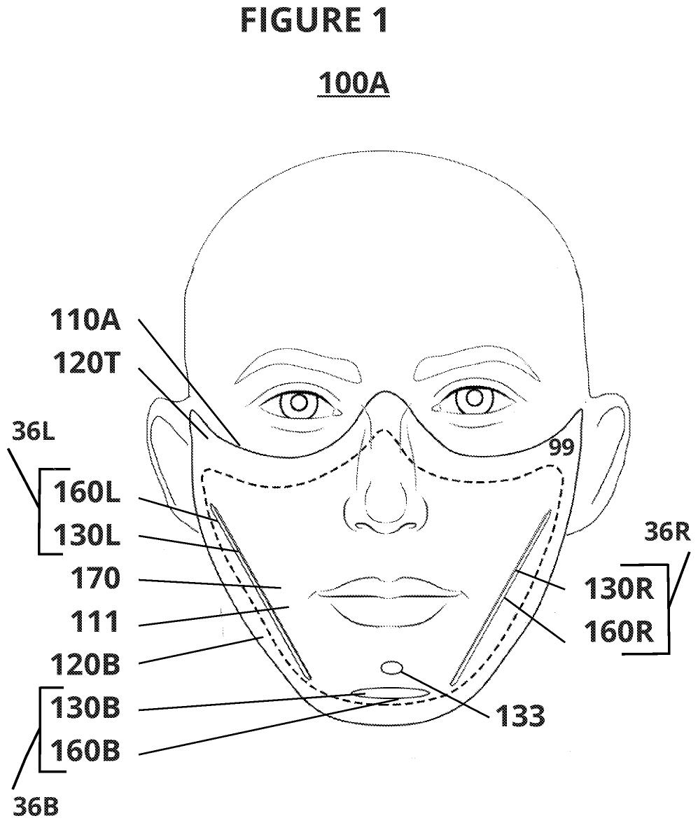

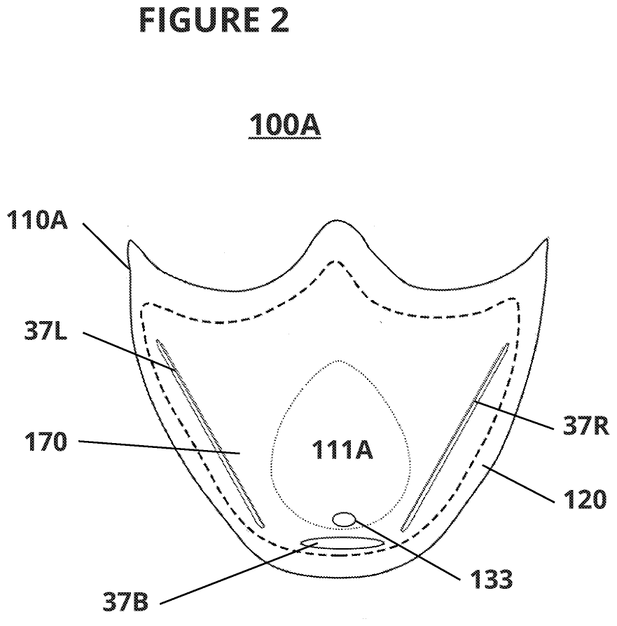

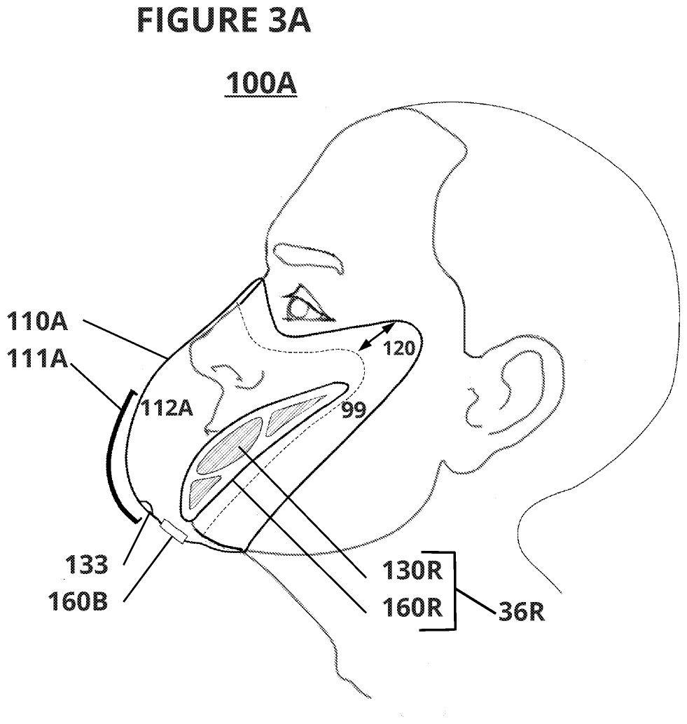

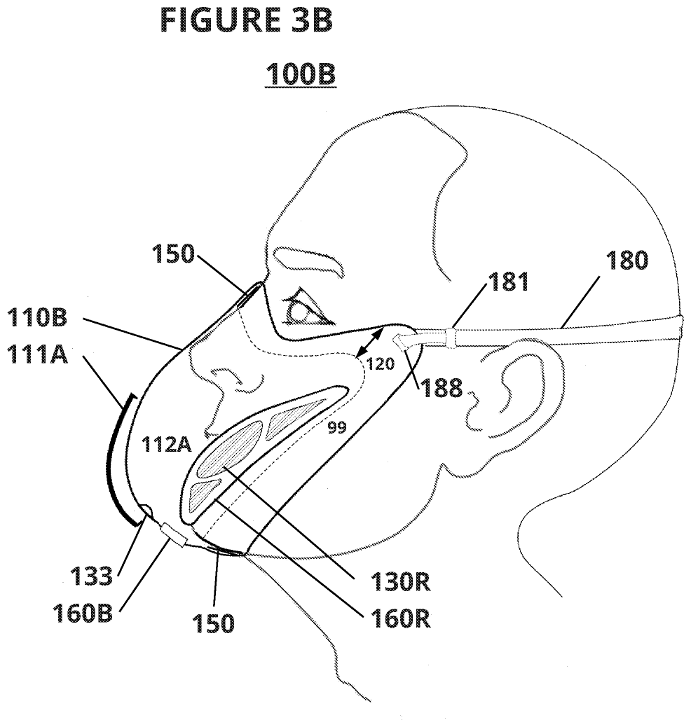

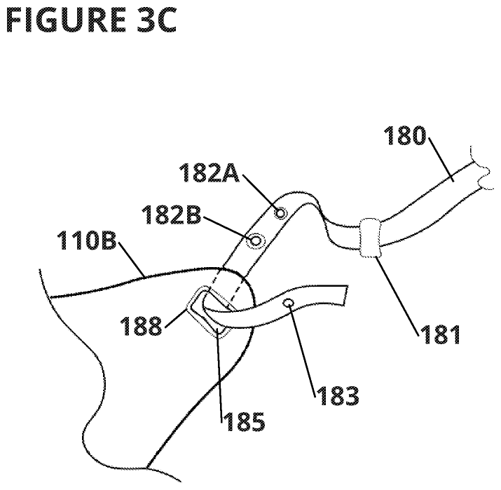

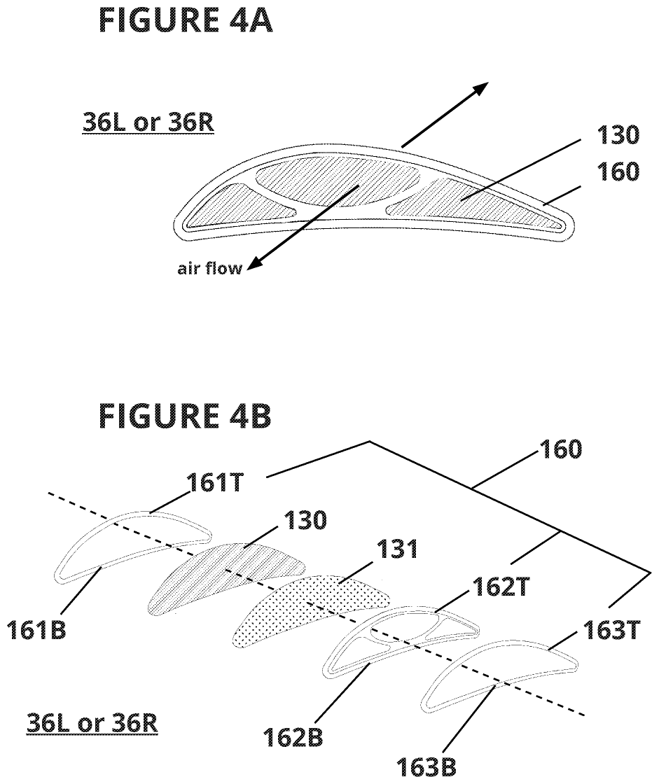

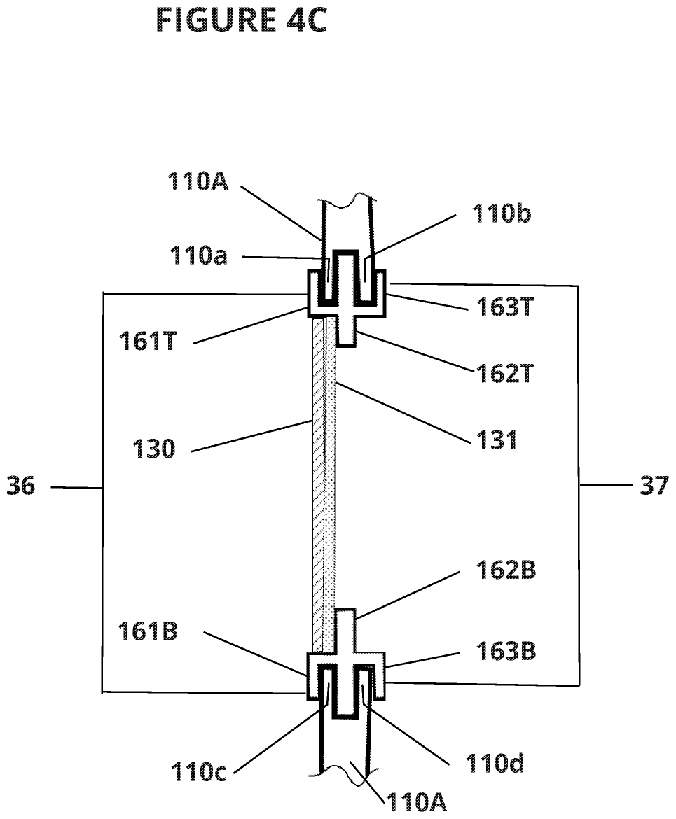

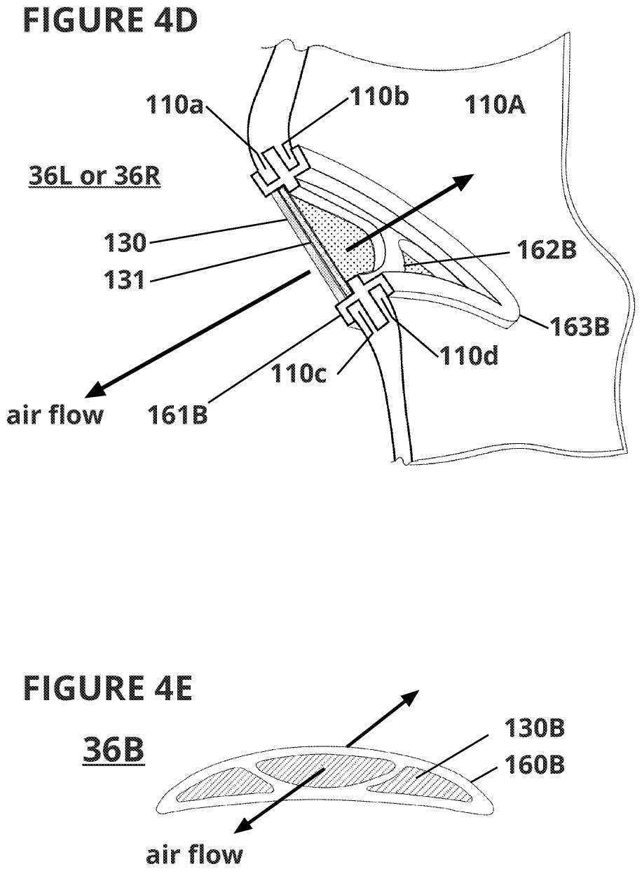

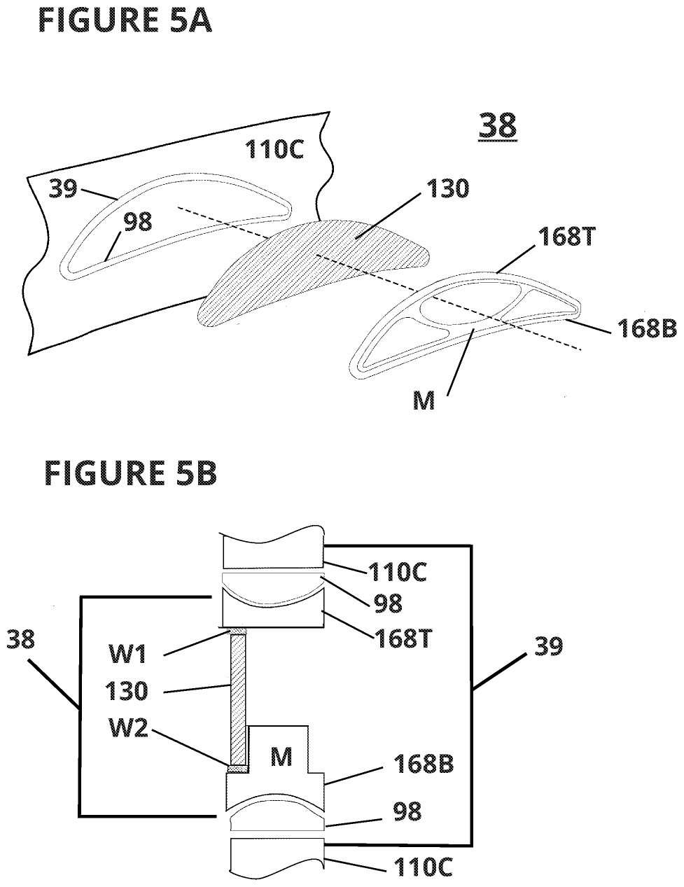

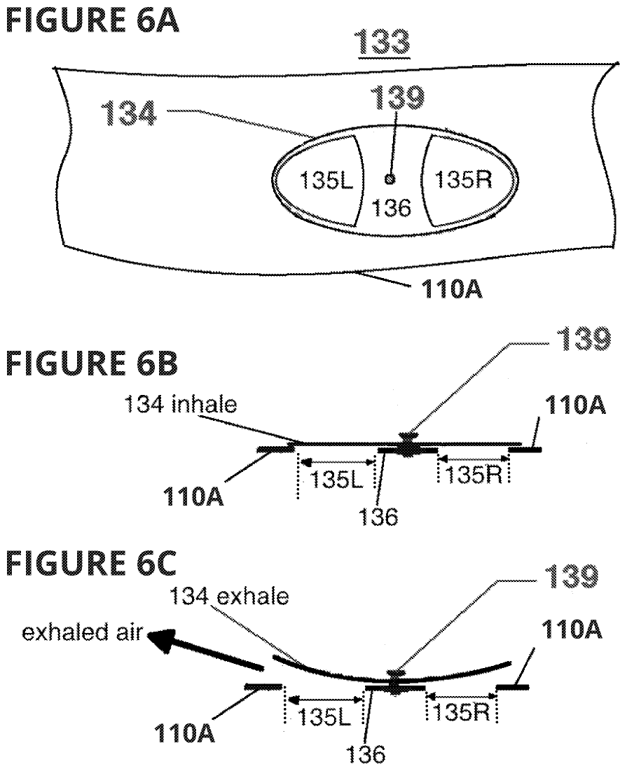

FIG. 1 is a frontal view of mask 100A being worn; FIG. 2 is a frontal view of mask 100A when not worn; FIG. 3A is a profile view of mask 100A being worn; FIG. 3B is a profile view of mask 100B being worn; FIG. 3C shows headband 180 of mask 100B; FIGS. 4A-4D are views of the side-of-face air filter cartridge used in mask 100A; FIG. 4E is a view of the bottom-of-face air filter cartridge used in mask 100A; FIGS. 5A-5B are views of the side-of-face air filter cartridge used in mask 100C; and FIGS. 6A-6C are views of the breathing valve used in mask 100A.

Mask 100A comprises
clear, flexible half-face shield 110A having
   underside 170 (the side closest to the wearer's skin),
   stiffened area 111;
   air filter cartridge openings 37B, 37L, 37R;
   perimeter border 120 comprising top border 120T and bottom border 120B,
clear removable adhesive 99 in perimeter border 120;
bottom-of-face air filter cartridge 36B comprising air filter 130B and air filter holder 160B;
side-of-face air filter cartridges 36L, 36R, respectively comprising air filters 130L, 130R and air filter holders 160L, 160R; and
breathing valve 133.

Shield 110A covers the mouth and nostril openings of its wearer. Shield 110A is formed of transparent and visually clear silicone, also called polysiloxane. In other variations, shield 110A may be slightly color-tinted as long as such tinting does not impede a third party's perception of the position of the wearer's lower face. Mask 100A flexes as the wearer moves her/his jaw.

When intended for adults, shield 110A has a size of about 4.5″×6″ (11×15 cm) and a thickness of about 1-2 mm, narrowing to 0.5-1.5 mm (about 0.05″) at its perimeter. When intended for children, shield 110A has a size of about of 20% to 50% smaller than when intended for an adult.

Shield 110A is typically made by molding silicone in an additive manner. However, the specific technique used is not critical, and any process that produces shield 110A as described herein is acceptable.

In one embodiment, shield 110A is made with a plaster mold which is sanded to the correct form, then a layer of resin and hardening agent is poured on top of the plaster to seal the surface of the mold. Silicone material is combined with a hardening agent allowing the silicone material to be fluid for approximately one hour. During this one-hour period, a first layer of silicone having a thickness of about 0.1 mm is poured on top of the layer of hardened resin. After the first layer of silicone becomes solid, an additional 4-5 layers of silicone are poured on top, with time for each layer to solidify, to create a total thickness of 0.5 mm-2.0 mm.

The mold has a cavity for each air filter cartridge opening 37B, 37L, 37R (see FIG. 2). The dimensions of openings 37B, 37L, 37R are sized so that the exterior of air filter cartridges 36B, 36L, 36R fit snugly into openings 37B, 37L, 37R.

To create stiffened area 111, additional layers of silicone are poured atop the solidified 4-5 layers to make area 111 more rigid, so area 111 will not collapse against the wearer's face, and will be able to enclose air bubble 112 between shield 110A and the wearer's face (see FIG. 3A). Area 111 has dimensions of about 4-6 cm by 7-8 cm. Air bubble 112 has dimensions of about 5-6 cm by 7 cm.

In another embodiment, the mold is made with a digital 3D model and 3D printer. The mold is shaped so that area 111 is automatically created as a more rigid portion of shield 110A. Silicone is injected into the mold using an injection-molding machine, to form shield 110A. The 3D model and 3D printed mold has the cavity for each opening 37B, 37L 37R integrated into the cast of the mold.

Perimeter border 120 of underside 170 of shield 110A is coated with clear adhesive 99 that is permanent relative to underside 170 and removable relative to the wearer's skin. When worn, adhesive 99 in perimeter border 120 forms air-tight seal 150 with the wearer's skin (see FIG. 3B). The width of perimeter border 120 is about 2-3 cm. The thickness of adhesive 99 in perimeter border 120 is about 0.1-0.2 mm. Adhesive 99 holds shield 110A of mask 100A to the wearer's face by hydrogel adhesion.

Clear adhesive 99 is a hydrogel or any type of emulsion adhesive that is non-toxic and safe for application to skin. A hydrogel is a network of cross-linked polymer chains that are hydrophilic. The properties of hydrogel are easy to “tune” based on the choice of molecules for their backbone and crosslinker structure, and/or by adding nanoparticles and microparticles. Polymer Science Inc. makes P-DERM hydrogel adhesives for fixation to the body, that can be loaded with cosmeceuticals, pharmaceuticals or antimicrobials, and are designed to be worn for up to seven days. They can be easily removed without residue, retain adhesion when exposed to perspiration, are highly flexible, and hypoallergenic. Hydrogels are suited for use for moist wound dressings, blister prevention, wearable devices and general fixation. Hydrogel passes heat from skin, making it more comfortable to wear.

Clear adhesive 99 is made of medical grade silicone hydrogel or emulsion adhesive, such as the silicone sold as gel tape for scar removal, for instance, AWD Medical Soft Silicone Gel Tape for Scar Removal, available at www.amazon.com and at awdmed.com/products. AWD Medical describes its product as made with soft, non-irritating, hospital grade silicone. The silicone gel layer assures gentle application and removal. It is designed for fixation of medical devices such as drains, tubes, probes, electrodes, and more. It holds firmly and is removable without pulling the skin. It is friendly to sensitive and fragile skin. It is ergonomic—soft, flexible, and has excellent adaptability. It sticks securely to the human body to form an occlusive, moist healing environment for scar softening and flattening. It is easy to apply and remove. It can be cut to the desired length and can be used with other dressings. It can be used repeatedly. It is breathable, water-resistant, and leaves no residue upon removal. It is latex-free and teflon-free, and safe for children and adults.

In other embodiments, adhesive 99 is made of Chitin infused with medicinal healing serum or therapeutic emollients, that function in similar manner to the silicone gel tape described above. Suitable ingredients for infusion include but are not limited to poly(acetylglucosamine), N-acetyl-D-Glucosamine (Chitosan) or chitin 120 and silicone or polysiloxane. Examples, all available at www.amazon.com, include:
a. Beauty wrinkle reducing therapy, silicone tape. TOPNaturePlus Facial Wrinkle Patches, Anti Face Wrinkle Pads, Facial Wrinkle Pads And Silicone Care Forehead And Reusable Under Eye Pads
b. Beauty wrinkle reducing therapy, silicone tape. TOPNaturePlus Facial Wrinkle Patches—Reusable Face Wrinkle Remover Strip—Repair Smoothing Tapes Facial Lines Filler for Upper Lip, Eye, Forehead, Mouth, Fine Lines
c. Scar healing silicone tape. Aroamas Professional Silicone Scar Removal Sheets for Scars Caused by C-Section, Surgery, Burn, Keloid, Acne, and more, Soft Adhesive Fabric Strips, Drug-Free, 5.7″×1.57″, d. Scar healing silicone tape: ScarAway Silicone Scar Sheets shrink, flatten and fade scars.

Moisture build up or fog condensation in mask 100A is eliminated by inhaling so that new air comes in and the flow of the air evaporates the moisture if it collects on underside 170. In some embodiments, an anti-fogging material, such as LOXIOL, EDENOL or EMEROX sold by Emery Oleochemicals, is added to the silicone of shield 110A for addition protection against fogging.

If the wearer of mask 100A also wears glasses, the glasses can be worn over mask 100A due to the thinness of shield 110A and the stability of mask 100A from air tight seal 150.

Air-tight seal 150 is formed between adhesive perimeter 120 and the wearer's epidermis. Air-tight seal 150 prohibits inhalants from leaking into the respiratory system of the user. Air-tight seal 150 is formed by hydrogel adhesion between clear adhesive 99 and the wearer's skin. Accordingly, the wearer's skin is not harmed by use of mask 100A. Rather, because of healing material embedded in adhesive 99, the wearer's skin health is improved by use of mask 100. In contrast, conventional masks are held against the user's skin by mechanical pressure, so that chafing occurs; such chafing can mature into wounds even after a few hours, referred to as “dermatitis”. Using mask 100A does not cause dermatitis, because there is no chafing due to mechanical pressure against the wearer's skin.

Adhesive perimeter 120 is an important feature of mask 100A, because (a) adhesive perimeter 120 is strong enough to keep mask 100A on the wearer's face, eliminating the need for uncomfortable ear loops or a head band to enable wearing of mask 100A, (b) adhesive 99 creates air-tight seal 150 with the wearer's face, protecting the wearer from airborne pathogens, (c) adhesive 99 is embedded with material that heals and moisturizes the wearer's skin, avoiding chafing, and (d) adhesive 99 is embedded with a gel that helps remove fogging, making mask 100A more useful and comfortable for its wearer.

D-shaped air filter cartridges 36B, 36L, 36R (generically, 36) removably insert into D-shaped openings 37B, 37L, 37R (generically 37), respectively, of shield 110A. Air filter cartridges 36 should be replaced when soiled on the outside surface, as the soiling may block air flow through the air filter. Washable basic (non-N95) air filter cartridges should be washed with soap and water or with alcohol, after about 10-15 hours of use, and should last for up to 100 washings. N95 air filter cartridges can be used for 12-16 weeks depending on environment.

Air filters 130L, 130R, 130B (generically, 130) are respectively in air filter holders 160L, 160R, 160B (generically, 160). In other embodiments of mask 100A, only one or two air filter cartridges, or more than three, are used. Air filters 130 function to filter inhaled (incoming) and exhaled (outgoing) air, and to permit heat and moisture exchange between air exterior to mask 100A and air bubble 112 that is interior to mask 100A.

Air filter cartridge 36 comprises air filter 130 and air filter holder 160. Side-of-face cartridges 36L, 36R each have dimensions of about 3.5-4″ (9-10 cm) by 0.5″ (10-15 cm) by 2-3 mm (for a small adult). Bottom-of-face cartridge 36B has dimensions of about 0.5-1.0 mm by 2.5-3.0 mm (for a small adult). Mask 100A typically is available in different sizes (large adult, small adult, small child), and the size of an air filter cartridge differs by mask size. Air filter 130 is permanently affixed to air filter holder 160 by sonic welding or other suitable process. In other embodiments, the air filter is removable from the air filter holder.

FIG. 3A shows, in profile, how air filter cartridge 36R fits in opening 37R (occluded) of shield 110A. Turning to FIG. 4A, the bidirectional air flow through side-of-face air filter cartridge 36R is shown, corresponding to inhalation and exhalation by the wearer of mask 100A. FIG. 4E corresponds to FIG. 4A, except it shows bottom-of-face air filter cartridge 36B. Other than size, the difference is that cartridges 36L, 36R have an elongated D-shape, while cartridge 36B has a symmetric D-shape. Cartridges 36L, 36R facilitate air flow horizontally across the wearer's face, while cartridge 36B facilitates air flow in an orthogonal direction to the wearer's mouth and nose.

FIG. 4B is an exploded view of air filter cartridge 36R. Air filter holder 160 comprises left panel 161, center panel 162 and right panel 163. Left and right panels 161, 163 are D-shaped loops, while center panel 162 is a D-shaped loop with an interior U-shaped member for added stability. Left and right panels 161, 163 snap onto either side of center panel 162, forming sturdy D-shaped air filter holder 160. To conform to the wearer's face, the D-shape is squashed on one side (near the wearer's mouth) and elongated on the other side (near the wearer's eyes), forming a streamlined D-shape, wherein the U-shaped interior opening of air filter holder 160 is adjacent to the wearer's mouth.

FIG. 4C is a partial cross section of shield 110A having air filter cartridge 36 inserted in opening 37 of shield 110A. Opening 37 fits snugly around the outside of cartridge 36. Opening 37 has a channel formed in its center, to accommodate center panel top 162T. The top edges of the channel in opening 37 form silicone prongs 110a, 110b; prong 110a fits between left panel top 161T and center panel top 162T, while prong 110b fits between center panel top 162T and right panel top 163T. The bottom edges of the channel in opening 37 form silicone prongs 110c, 110d; prong 110c fits between left panel bottom 161B and center panel bottom 162B, while prong 110d fits between center panel bottom 162B and right panel bottom 163B. Prong 110a and 110c form a continuous prong around one side of opening 37, while prong 110b and 110d form a continuous prong around the other side of opening 37. The prongs formed inside opening 37 serve to restrain air filter cartridge 36 inside opening 37.

FIG. 4D is a three-dimensional cutaway view corresponding to FIG. 4C.

To insert air filter cartridge 36 into opening 37, prongs 110a-d of shield 110A are carefully tucked between the panels of cartridge 36 using a tool with a small blunt point. To remove cartridge 36 from opening 37, prongs 110a-d are gently urged away from cartridge 36 using the tool with the small blunt point.

Air filter holder 160 is made of transparent polylactic acid (PLA). PLA is a plant-based degradable, bio-compostable and biodegradable plastic. PLA is a sustainable alternative to petroleum-based polymers because it reduces dependency on oil and provides solutions to the environmental concerns about the use of petroleum-based plastics. PLA exhibits many properties equivalent to or better than its petroleum-based counterparts. It has excellent stiffness, reasonable strength, excellent flavor and aroma barrier, as well as good grease and oil resistance. PLA has a low bioburden compared with other materials such as recycled wood pulp. This low bioburden profile precludes any heat drying needed to destroy microbes, allowing a “cleaner” and less process-intensive manufacturing method than traditional alternatives such as wood pulp. PLA has a very attractive biocompatibility profile and is resorbable and safe in terms of cytotoxicity versus synthetic fibers. Crystallization kinetics are different for PLA nonwovens when compared to petroleum-based polymers due to the processing parameters of higher pressure and extrusion temperatures.

Air filter holder 160 may be tinted to be a color such as black, white, silver or gold, so that the air filter cartridges are akin to jewelry for mask 100 and can be coordinated with the wearer's apparel. Alternatively, the color of air filter holder 160 may indicate the type of filter material 130. In other embodiments, holder 160 is made of silicone, or a rigid organic material, such as bamboo, treated to resist pathogens.

Between left panel 161 and center panel 162, there are located D-shaped air filter 130 and optional D-shaped activated carbon filter 131. Air filters 130, 131 are sized to fit snugly within air filter holder 160.

Air filter 130 is formed of a material suited to the type of protection desired by the wearer. It is expected that after a predetermined amount of use, air filter 130 will functionally degrade so that air filter cartridge 36 needs to be replaced. The material of air filter 130 can be chosen depending upon the type and toxicity of inhalants intended to be protected from in a given setting or circumstance, from at least the following materials: (a) a washable substance providing basic (non-N95) protection, that should be washed after about 10-15 hours of use, and can withstand at least 100 washings; (b) a HEPA-like material typically used in vacuum cleaners and air purifiers; and (c) conventional N95 material; (d) non-polypropylene base filter material which can also be used in other applications such as conventional masks and vacuum cleaner filters. Air filter 130 is sized to fit snugly inside air filter holder 160.

In one embodiment, air filter 130 is formed of HEPA-like air filter material. A High Efficiency Particulate Air (HEPA) filter removes from air passed through the filter at least 99.97% of particulates having a diameter of at least 0.3 micrometers (0.0003 millimeters). HEPA filters are widely used in high-end vacuum cleaners and air filters because they make it possible for individuals with medical conditions to easily breathe treated ambient air, rather than requiring air from an oxygen tank. The material for air filter 130 is air-spun poly very fine glass threads with a diameter of less than 1 micron (a micron is 0.00004 in or 0.001 mm), or non-polypropylene PLA thread fibers integrating silk, bamboo and or cotton fibers. The fine glass threads or non-polypropylene PLA thread fibers integrating silk, bamboo and or cotton fibers are tangled and compressed to form a filter mat. Because the individual threads are so microscopic, most of the mat consists of air. The openings in the mat are very small, generally less than 0.5 micron (0.00002 in, 0.0005 mm). When this material has a thickness of 0.10 in (2.5 mm), it interposes 2,500 layers of glass threads between the inside and outside of mask 100A. Air filter 130 has a thickness of about 0.13-0.35 mm. The material for air filter 130 optionally includes bactericide and/or essential oils, such as camphor and cinnamon, to assist in filtration and elimination of air pathogens and to make air smell better.

Air filter 130 can be made in a variety of colors to coordinate with the color of air filter holder 160, or to indicate the filtration strength of the air filter cartridge. Different filters will protect better against different conditions. For example, a home user protecting against pollen can use a washable basic air filter 130 without activated carbon filter 131, whereas a hospital worker can use a HEPA-like air filter 130 in combination with activated carbon filter 131.

Activated carbon filter 131 includes an activated carbon layer, a non-woven layer made of materials including but not limited to natural fibers of wool, silk, cotton and/or bamboo, and is sized to be similar to air filter 130 and fit snugly inside air filter holder 160. Activated carbon filters are well-known; their main purpose is to neutralize bad smells and volatile organic compounds (VOCs). VOCs make air harder to breathe and can endanger health. The layer of porous activated charcoal is able to adsorb gas molecules and large air particles. Adsorbing means that instead of soaking in the molecules stick to the carbon surface. The activated charcoal is so powerful it can trap cigarette smoke. Once stuck in the carbon, odors are neutralized. Carbon is a form of charcoal, either coconut, wood or coal. Carbon is carbon atoms that are connected together in a latticework, and goes through a process to become activated. The process makes the carbon more porous and therefore able to capture more molecules. The carbon may also be chemically processed to improve its ability to filter certain air pollutants. One gram of activated carbon can have a surface area exceeding 500 square meters. When an activated carbon air filter needs to be replaced or cleaned it emits a foul odor.

FIGS. 6A-6C are views of optional breathing valve 133 comprising clear flap 134 and fastener 139 operating in conjunction with openings 135L, 135R in shield 110A. Breathing valve 133 has dimensions of about 1″×0.6″ (2 cm×1.5 cm). Optional breathing valve 133 is located next to the chin of the wearer, inside perimeter 120. Preferably, breathing valve 133 automatically closes during inhalation and automatically opens during exhalation, facilitating reduction of heat and moisture in air bubble 112, and reducing fogging. Air flows into air bubble 112 through the air filters 130, and out of air bubble 112 through breathing valve 133 and air filters 130.

As shown in FIG. 6A, shield 110 of mask 100 has left opening 135L and right opening 135R approximately in the middle chin area of the wearer, forming bridge 136. That is, bridge 136 is the portion of shield 110 between openings 135L and 135R. Openings 135L, 135R are shown as tongue-shaped, but other shapes are also suitable. Flap 134 is placed so that it extends across openings 135L, 135R. The size of flap 134 is slightly larger than the area formed by openings 135L, 135R and bridge 136. Flap is shown as having an elliptical shape, but other shapes are also suitable. Clear flap 134 is made of a material that flexes under pressure such as silicone or Flap 134 is on the outside of shield 110, that is, the side of shield 110 that faces the external environment, not the wearer's face.

Fastener 139 is located at the center of bridge 139, and serves to hold flap 134 against shield 110. Fastener 139 may be mated male and female snaps, or a prong with securing heads on each end, or other appropriate device. Fastener 139 is made of a rigid material such as plastic or metal, and has a diameter of about 0.5″ (10-12 mm).

FIGS. 6B and 6C show operation of breathing valve 133. As shown in FIG. 6B, when the wearer of mask 100A inhales, air pressure keeps flap 134 in its default flat position, covering openings 135L, 135R, so that air does not pass through valve 133. As shown in FIG. 6C, when the wearer of mask 100A exhales, air pressure forces flap 134 into a flexed position, exposing openings 135L, 135R, so that outgoing air passes through valve 133. In the interval after exhalation and before inhalation, flap 134 returns to its preferred flat (unflexed) position, protecting the wearer of mask 100A from pathogens in the ambient air. While air filters 130 operate during inhalation and exhalation, it will be appreciated that they are most active during inhalation, because most exhaled air exits via breathing valve 133.

In other embodiments, breathing valve 133 is constructed differently, while serving to block air flow during inhalation and permit air flow during exhalation.

Putting on mask 100A will now be discussed.

The wearer must wash her/his hands before putting on mask 100A, or use sterile gloves, to prevent contaminating underside 170 of shield 110A with pathogens. In a variation, the wearer can omit hand-washing but must ensure the interior surfaces of mask 100A do not get contaminated before wearing. The wearer should cleanse their facial skin immediately before placing mask 100A on their facial skin. Adhesive 99 in perimeter 120 is hypoallergenic. Mask 100A is typically shipped with a protective layer (not shown) over adhesive 99 in perimeter 120, and this protective layer must be removed prior to first use of mask 100A.

The wearer positions mask 100A by aligning the nose bridge of mask 100A with the wearer's own nose bridge and chin. The wearer holds mask 100A by cupping the exterior of shield 110A and placing underside 170 of shield 110A against the wearer's facial skin, and pressing gently yet firmly along the perimeter of shield 110A to form air-tight seal 150.

Mask 100A may be worn for 8-12 hours per day without causing chafing or irritation.

Cleaning mask 100A will now be discussed.

The inner and outer surfaces of mask 100A can be cleaned in any of the following ways:
a. after removing air filter cartridges 36, or being careful to keep them dry, mask 100A is washed and sanitized with alcohol or any other surface detergent for the removal of potential pathogens that can linger on the surface;
b. put in an autoclave for sterilization, after removing air filter cartridges 36;
c. put in a microwave oven on high for 3 minutes to sterilize mask 100A for reuse, after removing air filter cartridges 36.
   Medical personnel in hospitals should replace air filter cartridges 36 at least daily, and preferably each time that mask 100A is used in a sterile operating room. Home users should replace air filter cartridges 36 preferably after two days of use, but certainly after a week of use.

Advantages of mask 100A include, but are not limited to, the following:
1. Non-interference with facial socialization and facial communication. Because mask 100A is transparent, it enables (a) deaf persons to lip read the wearer of mask 100A, (b) perception of the social cues from the lower face of the wearer of mask 100A, such as humor (smiles), skepticism (mouth slightly opened), shock (mouth widely opened), discomfort (mouth opened to bare teeth), or anger (mouth sneering or snarling) and (c) identification of the wearer of mask 100A by face.
2. Comfort. The adhesive perimeter of mask 100A, in combination with its light weight and flexibility, eliminates the need for ear loops or behind-the-head ties, thereby eliminating chafing or discomfort from such ear loops or ties.
3. Health-promotion. Because the adhesive in the perimeter of mask 100A is used for skin protection and healing, the wearer's skin may be improved by extended wearing of mask 100A.
4. Protection for the wearer against airborne pathogens. Mask 100A serves as an air barrier, protecting the wearer's nose and mouth, and thus respiratory system, from airborne pathogens such as dust, pollution, bacteria, viruses, pollen, thereby protecting against diseases such as Covid-19, Ebola, Cholera, Ecoli, and Zika.
5. Protection for third parties against airborne pathogens from the wearer of mask 100A. First, to the extent that exhaled air from the wearer of mask 100A enters the environment via air filters 130, the wearer's exhalations are automatically filtered, making the environment safer for others. Second, even if the wearer's exhaled air enters the environment via breathing valve 133, the direction of the exhaled air is not directly at a third-party facing the wearer of mask 100A, making it harder for pathogens from the wearer to reach the third party.
6. Easy to apply. No particular skill or training is required to apply mask 100A. Mask 100A can be applied kinesthetically, by simply running one's finger around the perimeter to ensure an entire seal. Mask 100A can be applied visually, by the wearer inspecting their appearance in a mirror. A third-party can apply mask 100A to the wearer.
7. Easy to remove. No particular skill or training is required to remove mask 100A. Either the wearer or a third party gently breaks the perimeter seal at one or more points, then gently lifts the rest of the perimeter from the wearer's face.
8. Reusable. Mask 100A is resilient, reusable, and recyclable.
9. Easy to clean. No special equipment or preparations are needed to clean mask 100A. Mask 100A can be washed or wiped with soap and water, or sterilized in a microwave or autoclave.
10. Continued potency. When air filter cartridges 36 are replaced according to the recommended schedule, mask 100A is always potent against pathogens.
11. Versatile. Because air filter 130 can be chosen to best match the conditions expected by the user, mask 100A is easily optimized for a variety of situations.
12. Durable. Because shield 110A is impervious to water, splashing or spitting from a third party does not harm mask 100A, as occurs with cloth or paper masks, and the wearer does not need to immediately change to a new mask but instead can continue with their activity.

Table 1 is a chart comparing mask 100A to other protective face masks. No other face masks are wearable without ear loops or head ties. Polypropylene is a thermoplastic polymer considered safe for filters, but which degrades slowly after disposal. Bisphenol A (BPA), an estrogen-mimicking chemical that can interfere with body growth, cell repair, fetal development, human energy levels and reproduction, is added to many containers, cans used for canned food, and hygiene products; its use has been restricted in the EU, Canada, China and Malaysia, but there are no American federal restrictions. The problems with masks needing electricity include that creating electricity causes CO2 emissions, polluting the environment.

TABLE 1

| | | Conventional masks | | | | |
|---|---|---|---|---|---|---|
| | Mask | | 3M | | UV | 3M S6000 |
| | 100A | Surgical | Respirator | Cloth | Respirator | Respirator |
| Transparent | YES | — | — | — | YES | — |
| Reusable | YES | — | — | YES | YES | YES |
| Air-tight seal | YES | — | YES | — | YES | YES |
| No poly- | YES | — | — | YES | — | — |
| propylene | | | | | | |
| No BPA | YES | — | — | YES | — | — |
| No pressure | YES | — | — | — | — | — |
| against face | | | | | | |
| No foggy | YES | — | — | — | YES | YES |
| glasses | | | | | | |
| N95 or better | YES | — | YES | — | YES | YES |
| Needs | — | — | — | — | YES | YES |
| electricity | | | | | | |

Mask 100B (not shown) comprises mask 100A with optional removable headband 180. FIG. 3B is a profile view of mask 100B being worn, and FIG. 3C shows headband 180 of mask 100B.

Headband 180 serves to keep mask 100B on top of the wearer's head even if the wearer breaks air-tight seal 150, such as to eat or drink or scratch an itch on their face, saving the trouble of the wearer remembering where s/he put her mask after taking it off. Alternatively, headband 180 enables mask 100A to be pulled down to the neck of the wearer and rest against the wearer's upper chest, with mask 100A folded to keep underside 170 clean and germ-free. Headband 180 is not needed to keep mask 100B on the wearer's face via mechanical pressure because adhesive perimeter 120 keeps mask 110C on the wearer's face, so headband 180 should be somewhat loose, reducing the chance of pain from wearing the headband.

Mask 100B has face shield 110B corresponding to face shield 110A, except that face shield 110B has left upper opening 188L and right upper opening 188R (generically, upper opening 188). The edges of opening 188 may be formed as slightly thicker silicone to improve the sturdiness of opening 188.

Headband 180 is a strip of elastic having a length chosen to fit around the wearer's head, and having a width of about 0.25-0.5″ (5-12 mm). The ends of headband 180 each have male snap 183 and several female snaps 182 (two female snaps 182A and 182B are shown). To attach headband 180, the ends of headband 180 are respectively threaded through upper openings 188 of mask 100B, then male snap 183 is pushed into one of female snaps 182, and slidable cover 181 is pushed over the snap formed by male and female snaps 182, 182. To remove headband 180, the male and female snaps are separated at each end of headband 180, then the ends of headband 180 are gently unthreaded through upper openings 188 of mask 100B.

Instead of, or in addition to, headband 180, mask 100B may optionally be provided with ear loops (not shown) to enable mask 100B to loosely hang from the wearer's ears when the wearer is not wearing the mask, such as a rest break between periods of wearing mask 100B.

Mask 100C (not shown) is similar to mask 100A with different air filter cartridges than mask 100A and a different way of connecting the air filter cartridges to the silicone shield. For brevity, only differences are discussed. FIGS. 5A-5B are views of D-shaped air filter cartridge 38 used in mask 100C. Air filter cartridge 38 is about the same size as air filter cartridge 36 of mask 100A.

FIG. 5A shows an exploded view of air filter cartridge 38 that fits into opening 39 of shield 110C of mask 100C. Opening 39 is slightly larger than the outside of air filter cartridge 38. A strip of removable flexible clear adhesive 98 is located on the inside of opening 39. Adhesive 98 is permanently attached to opening 39. Adhesive 98 removably attaches to the outside perimeter of air filter cartridge 38. Adhesive 98 may be the same material as used for adhesive 99 in mask 100A. Air filter cartridge 38 comprises air filter holder 168—having top 168T and bottom 168B—and air filter 130, and may include optional activated charcoal filter 131. Air filter holder 168 is similar to center panel 162 of air filter cartridge 36 of mask 100A.

FIG. 5B shows a cross section through shield 110C when air filter cartridge 38 has been inserted into opening 39. The thickness of strip 98, about 0.5-1.0 mm, occupies the difference in size between the outer perimeter of air filter cartridge 38 and opening 39. The outer perimeter of air filter holder 168 may be formed with a U-shaped channel, improving the adhesion between the strip of adhesive 98 and cartridge 38. The perimeter of air filter 130 is sonic welded to the inner perimeter of air filter holder 168, indicated as welds W1 and W2.

To insert air filter cartridge 38 into opening 39, adhesive 98 is pressed against the outer perimeter of air filter holder 168. To remove cartridge 38 from opening 39, adhesive 98 is gently urged away from the outer perimeter of air filter holder 168 until they are separated.

### Full-Face Mask

Mask 200 is a full-face mask having embodiments 200A and 200B. Mask 200A is similar to mask 100A, except that mask 200A is full-face and has forehead air cartridge 36F, headlamp 250 and glasses gate 240. Mask 200B is similar to mask 200A but with a different embodiment of its glasses gates. For brevity, only differences between embodiments will be discussed. Mask 200 is held to the wearer's face solely by clear flexible adhesive embedded with healing material, not by a headband or ear loops.

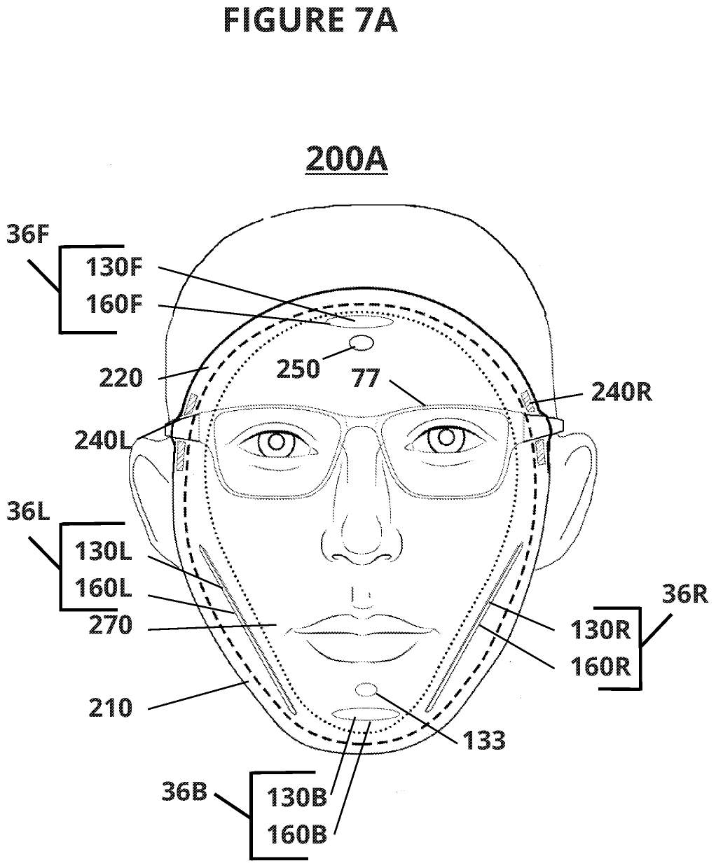

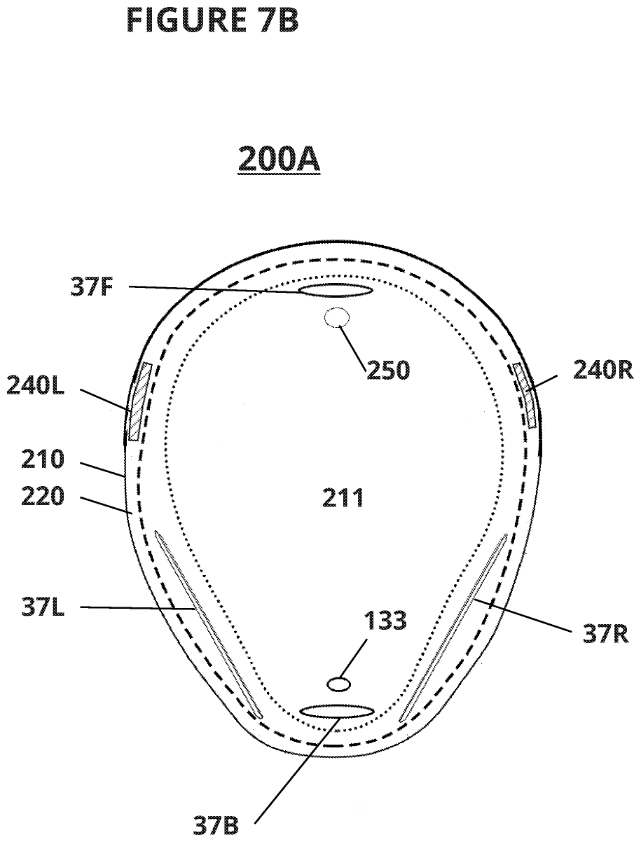

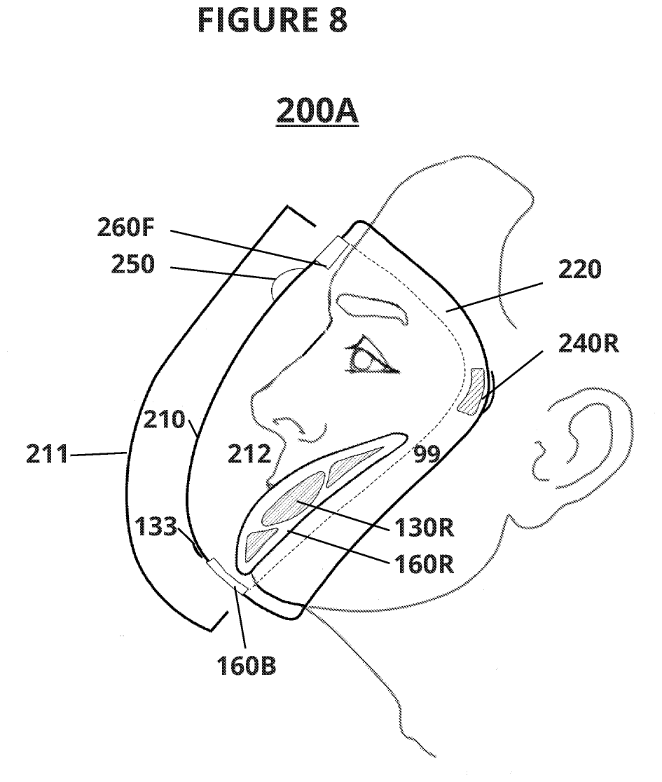

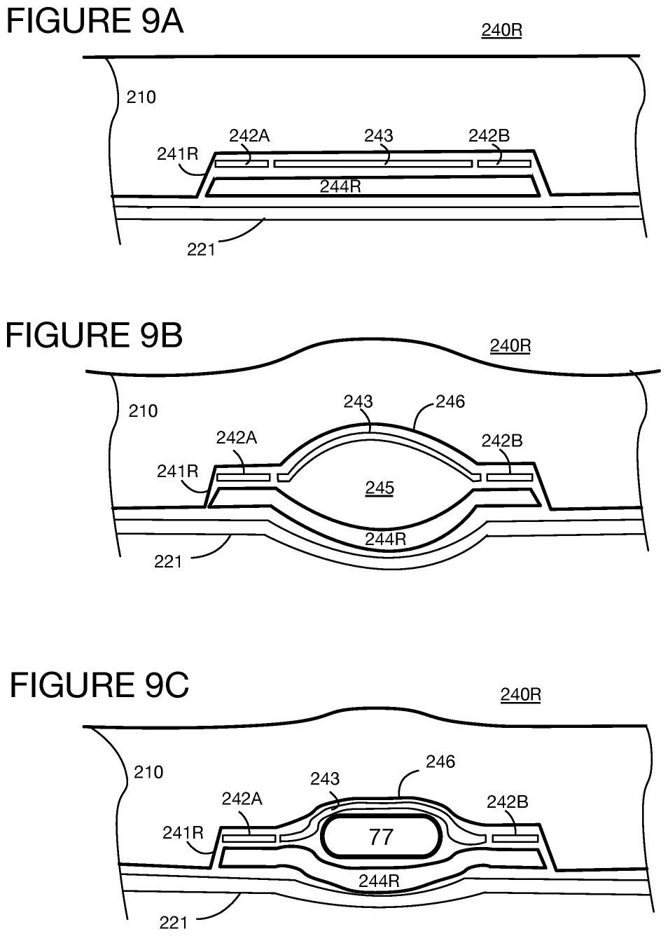

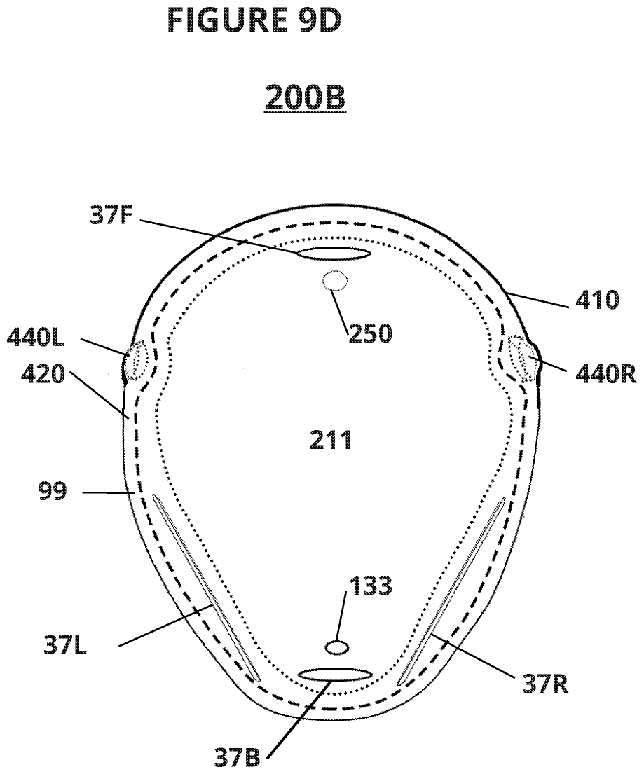

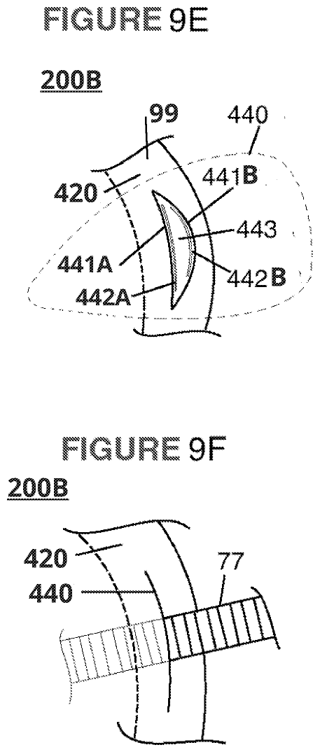

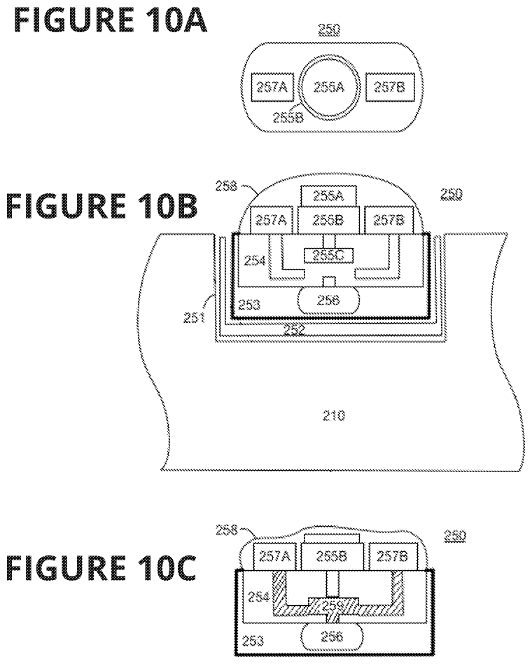

FIG. 7A is a frontal view of mask 200A being worn; FIG. 7B is a frontal view of mask 200A when not worn; FIG. 8 is a profile view of mask 200A being worn; FIGS. 9A-9C are views of glasses gate 240 of mask 200A; FIG. 9D is a frontal view of mask 200B when not worn; FIGS. 9E-9F are views of glasses gate 440 of mask 200B; and FIGS. 10A-10C are views of headlamp 250.

Mask 200A comprises
clear, flexible full-face shield 210 having
   underside 270 (the side closest to the wearer's skin),
   stiffened area 211;
   air filter cartridge openings 37F, 37B, 37L, 37R;
   perimeter border 220,
clear removable adhesive 99 in perimeter border 220;
forehead air filter cartridge 36F comprising air filter 130F and air filter holder 160F;
bottom-of-face air filter cartridge 36B comprising air filter 130B and air filter holder 160B;
side-of-face air filter cartridges 36L, 36R, respectively comprising air filters 130L, 130R and air filter holders 160L, 160R;
breathing valve 133;
glasses gates 240L, 240R; and
headlamp 250.

Shield 210 is similar to shield 110A except shield 210 is oval shaped, and functions to cover the eyes, nose and mouth of the wearer. When intended for adults, shield 210 has a size of about 4-5.5″ by 6-7″ and a thickness of about 0.5-2.0 mm. When intended for children, shield 210 has a size of about of 20% to 50% smaller than when intended for an adult. Shield 210 has air filter cartridge openings 37F, 37B, 37L, 37R for receiving air filter cartridges 36F, 36B, 36L, 36R, respectively. Air filter cartridge 36F is D-shaped and similar to air filter cartridge 36B, discussed above.

Area 211 of mask 200A is similar to area 111A of mask 100A except that area 211 begins just inside adhesive perimeter area 220. Area 211 has a size of about 4-6 cm by 7-8 cm. Air bubble 212 is similar to air bubble 112, except that air bubble 212 covers the wearer's entire face inside adhesive perimeter 220.

Glasses gates 240L, 240R are mirror images of each other. In FIG. 7A, the wearer of mask 200A is shown as wearing glasses 77. Glasses 77 are enclosed in air bubble 212. The left and right arms of glasses 77 respectively extend through glasses gates 240L and 240R, located at the edge of shield 210. Glasses gates 240L and 240R are collectively referred to as glasses gate 240, discussed below.

FIGS. 9A-9C are not to scale, and are drawn with elements separated for comprehension clarity.

FIG. 9A shows glasses gate 240R when unused, i.e., when the wearer of mask 200A does not wear glasses. A side view of shield 210, in the area of the wearer's right temple is shown. The thickness of shield 210 in the area of glasses gate 240R corresponds to its height in FIG. 9A, and is about 1.0-1.5 mm.

Channel 241R functions to essentially hide glasses gate 240R when the wearer does not wear glasses.

Channel 241R is formed at the edge of shield 210, having a thickness (vertical dimension in FIG. 9A) of about 0.3-0.4 mm, a length (horizontal dimension in FIG. 9A) of about 3-4 cm, and a width (the dimension extending across the wearer's face, orthogonal to the page of FIG. 9A) approximately equal to the width of adhesive perimeter 220 of mask 200A.

Channel 241R has permanent adhesive areas 242A, 242B at the edges of channel 241R, and reusable adhesive area 243 in the middle of channel 241R. The reusable adhesive in area 243 permanently adheres to channel 241R. The permanent and reusable adhesives are flexible and clear; the reusable adhesive may be adhesive 99 discussed above, while the permanent adhesive is similar. Adhesive areas 242A, 242B, 243 have a height (thickness) of about 0.1-0.25 mm and a width equal approximately equal to the width of adhesive perimeter 220 of mask 200A. Adhesive areas 242A, 242B are each about 15% of the length of channel 241R, while adhesive area 243 is about 70% of the length of channel 241R

Band 244R is formed of the same clear flexible silicone as mask 200A, and has a thickness (vertical dimension in FIG. 9A) of about 0.1-0.2 mm, a length (horizontal dimension in FIG. 9A) and a width (the dimension extending across the wearer's face, orthogonal to the page of FIG. 9A) approximately equal to the length and width of channel 241R. Note that the thickness of band 244R plus the thickness of adhesive area 243 is approximately equal to the thickness of channel 241R.

The side of band 244R that abuts the wearer's skin is covered with adhesive 221 used in perimeter 220. Adhesive 221 permanently adheres to shield 210 and band 244R, and removably adheres to the wearer's face. Adhesive 221 may be the same as adhesive 99 of mask 100A. Adhesive 221 has a thickness (vertical dimension in FIG. 9A) of about 0.1-0.2 mm.

The side of band 244R opposite the wearer's skin and abutting shield 210 is permanently retained on its outer edges by adhesive in areas 242A, 242B in channel 241R, and removably retained by adhesive in area 243 in channel 241R.

When the wearer of mask 200A does not wear glasses, transparent glasses gate 240R is contained entirely in channel 241R, effectively being hidden.

When the wearer of mask 200A wears glasses 77, s/he gently separates band 244R from shield 210 forming opening 245, then threads the arm of glasses 77 through opening 245, places mask 200A on his/her face for positioning with respect to glasses 77, then gently pinches shield 210 and band 244R to form an air-tight seal around glasses arm 77. Similar activity occurs for glasses gate 240L. Finally, the wearer seals mask 200A to the wearer's face by pressing along perimeter 220.

To remove glasses 77 from mask 200A, the wearer removes mask 200A from their face, and gently separates band 244R from shield 210 forming opening 245, unthreads the arm of glasses 77 through opening 245, then gently pinches shield 210 and band 244R back to their flat states of FIG. 9A, effectively hiding glasses gate 240R. Similar activity occurs for glasses gate 240L.

FIG. 9B shows glasses gate 240R when it is separated to form opening 245. Band 244R is stretched to form half of the perimeter of opening 245, and shield 210 is pushed away from band 244R to form the other half of the perimeter of opening 245. Bump 246 is created at the middle of channel 241R, atop opening 245. Shield 210 deforms outwards generally parallel to bump 246.

FIG. 9C shows glasses gate 240R when an arm of glasses 77 has been threaded through gate 240R and the arm has been sealed within glasses gate 240R. Glasses arm 77 typically has a thickness (vertical dimension in FIG. 9C) of about 0.5-2.0 mm. Notice that the middle of channel 241R is deformed upwards by the thickness of glasses arm 77, so bump 246 persists from FIG. 9B albeit with a smaller height; bump 246 ends, in the direction orthogonal to the paper of FIG. 9C, at area 211 where shield 210 is raised upwards from the wearer's face creating air bubble 212. Importantly, the bottom of glasses gate 240R is mostly flat, avoiding an uncomfortable projection and/or pressure into the wearer's temple.

Headlamp 250 is located in the center of the forehead area of mask 200A.

FIG. 10A shows a frontal view of headlamp 250, as seen by a third party looking at mask 200 while it is worn by a mask wearer. Headlamp 250 has dimensions of about 2×3×1 cm. Headlamp 250 includes an on/off switch having button 255A and housing 255B (collectively referred to as on/off switch 255), and light emitting diodes (LEDs) 257A, 257B. In other embodiments, from one to eight LEDs are used. When on/off switch 255 is in its off position, the LEDs do not provide illumination. When on/off switch 255 is in its on position, LEDs 257A, 257B provide illumination.

FIG. 10B is a cutaway view of headlamp 250 inserted in shield 210 in its off position. When shield 210 is formed, it includes channel 251 for receiving headlamp 250. Clear permanent adhesive 252 is placed in channel 251, then headlamp 250 is pressed into channel 251 so as to be permanently affixed within channel 251.

LEDs 257A, 257B, on/off switch 255, and lithium-ion battery 256, and their respective electrical wiring, are affixed to printed circuit board (PCB) 254. PCB 254 is snap-fit into housing 253. Clear flexible barrier 258 is attached to housing 253 over the LEDs and switch, to provide a watertight barrier for headlamp 250 so that it can be used in an autoclave, and to facilitate wiping fluid, such as a patient's spit, from headlamp 250. Clear flexible barrier 258 is formed of silicone.

Button 255A is in its off position, so electrical connector 255C extending from the bottom of button 255A, is unable to reach the electrical wiring for LEDs 257A, 257B and battery 256.

FIG. 10C is a cutaway view of headlamp 250 in its on position. Button 255A is depressed within housing 255B, causing barrier 258 to somewhat collapse, and causing electrical connector 255C to come into electrical communication with the electrical wiring for LEDs 257A, 257B and battery 256, forming circuit 259 enabling current to flow, so that LEDSs 257A, 257B provide illumination.

In some embodiments (not shown), other components such as a resistor and a capacitor are used in circuit 259.

In other embodiments, headlamp 250 includes a “remote epitaxy”-like micro circuit, created by growing thin films of semiconducting material on a large, thick wafer of the same material, which is covered in an intermediate layer of graphene, enabling a flexible circuit, integrated with the silicone like layer, integrate flexible, multifunctional electronic devices for lighting or sensors, with embedded LEDs for lighting the area the user is viewing.

In other embodiments (not shown), mask 200A includes a hearing device such a Bluetooth ear listening device and or microphone for communicating with another user and/or other people using the same device and face mask through Bluetooth enabled communication.

Mask 200B is similar to mask 200A but with a different embodiment of its glasses gates. For brevity, only differences are discussed.

FIG. 9D shows mask 200B having glasses gates 440L and 440R (generically, 440), instead of 240L and 240R of mask 200A. Each of glasses gate 440L, 440R is simply a slit in shield 410 inside adhesive perimeter 420. Shield 410 is slightly thinner around glasses gate 440, and perimeter 420 is widened along glasses gate 440. Area 211 indents inwards along glasses gate 440.

The slit of glasses gate 440 has removable clear flexible adhesive permanently attached to its inner perimeter. When the wearer is not wearing glasses, there are only slight bulges in perimeter 420 at the wearer's temples. To wear glasses under mask 200B, the glasses gate is opened, the arm of the glasses is threaded through the opening, the mask and glasses are positioned on the wearer's face, then perimeter 420 is pressed against the wearer's face and pinched together around the arm of the glasses, forming an air-tight seal. To remove, the mask and glasses are removed from the wearer's face by gently separating perimeter 420 from the epidermis, then gently opening the glasses gates 440L, 440R and unthreading the arms of the glasses from the openings in the glasses gates.

FIG. 9E shows glasses gate 440 opened. Removable adhesive 99 is permanently affixed to the underside of mask 200B in perimeter area 420. Glasses gate 440 has left side 441A and right side 441B, forming opening 443 when separated. Strip 442A of removable adhesive 99 is permanently affixed to left side 441A, and strip 442B of removable adhesive 99 is permanently affixed to right side 441B. Left and right sides 441A, 441B have a length of about 2-3 cm. Strips 442A, 442B have a thickness (horizontal dimension in FIG. 9E) of about 0.1-0.2 mm. The width (horizontal dimension in FIG. 9E) of perimeter area 420 along glasses gate 440 is expanded from about 2 cm to about 3 cm.

FIG. 9F shows glasses gate 440 closed around glasses arm 77. Because of the thinness of shield 410 in around glasses gate 440, and the flexibility of shield 410 and the strength of adhesive 99, shield 410 is warped into the configuration needed to form an air-tight seal around glasses arm 77, personalizing mask 200B for its wearer's needs.

Advantages of mask 200 include, but are not limited to, the advantages discussed above for mask 100, and further:
Protection is improved, since the wearer's eyes have a physical barrier, shield 210, separating them from the ambient environment, isolating the wearer's eyes; and
Headlamp 250 provides extra light when the wearer of mask 200 is doing detail work, such as a dentist or hygienist working in a patient's mouth.

### Goggles and Half-Face Mask

Goggles 300 are an upper-half-face mask having embodiments 300A and 300B, similar to half-face mask 100A. Only differences are discussed, for brevity.

Goggles 300 are shown as worn with half-face mask 100, although goggles 300 may be worn by themselves, without half-face mask 100. Half-face mask 100D is similar to half-face mask 100A; the difference is that mask 100D has a larger stiffened area 111D than stiffened area 111A of face mask 100A, resulting in a larger air bubble 112D.

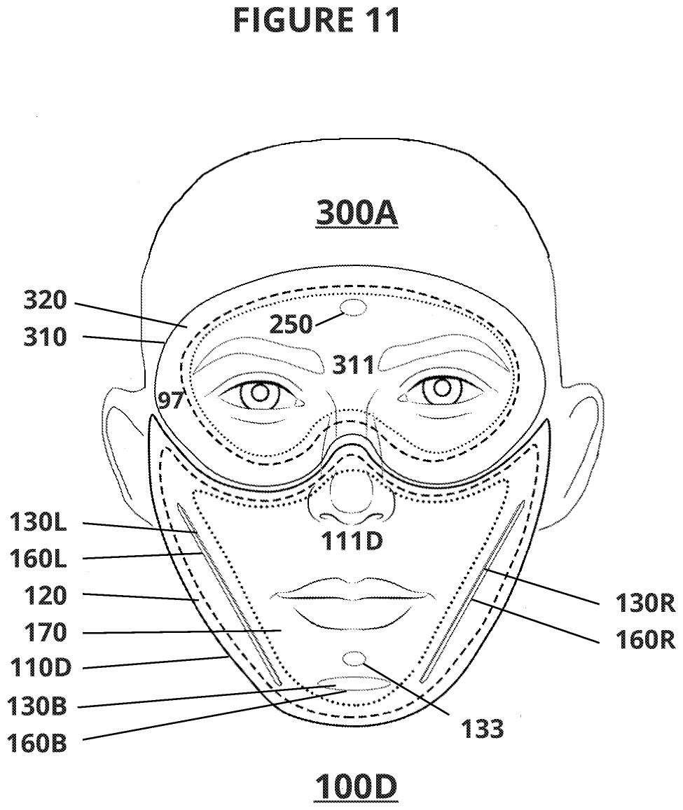

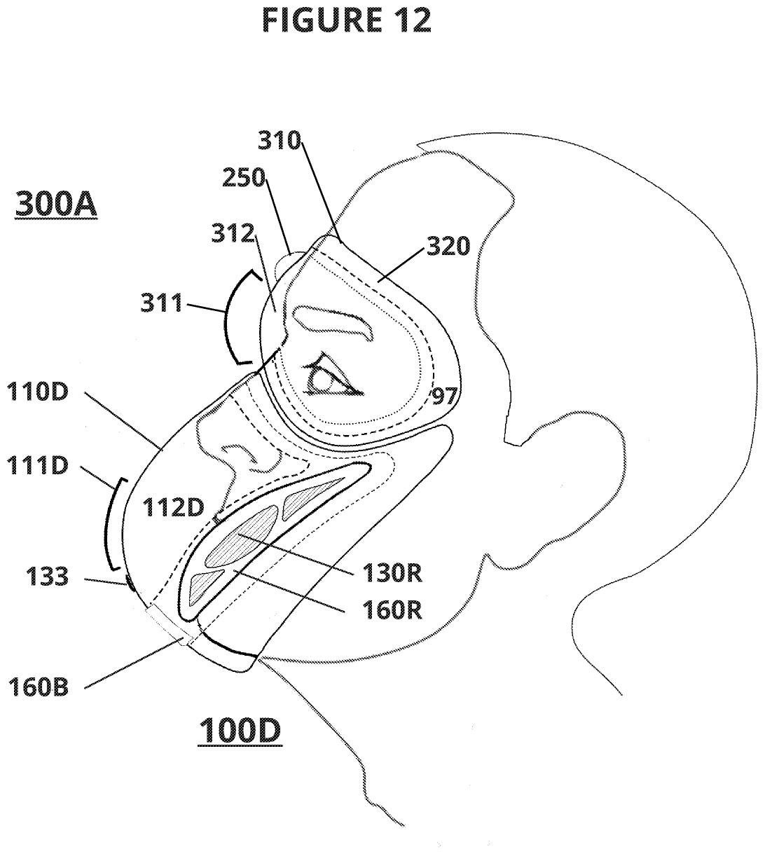

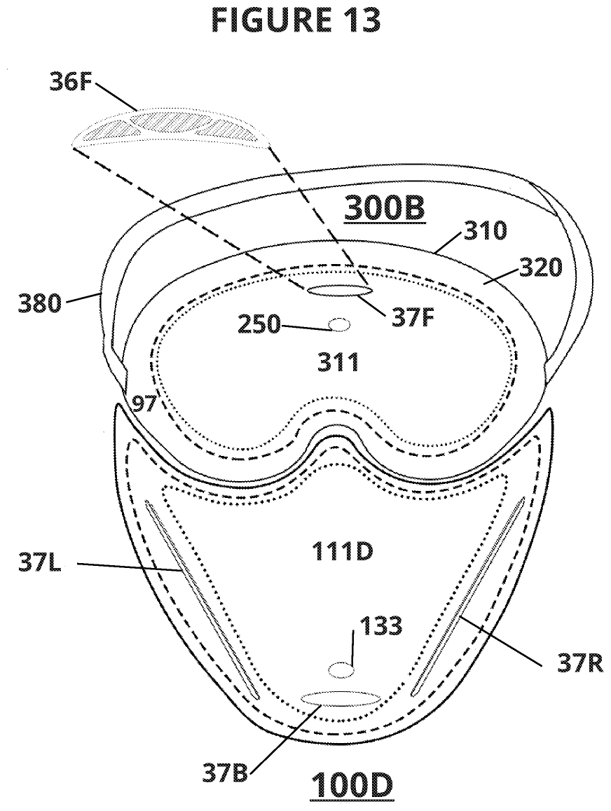

FIG. 11 is a frontal view of goggles 300A and half-mask 100D being worn simultaneously; FIG. 12 is a profile view of goggles 300A and half-mask 100D being worn simultaneously; and FIG. 13 is a frontal view of goggles 300B and half-mask 100D when not worn.

Goggles 300A have shield 310, similar to shield 110A except that shield 310 functions to cover the eyes of the wearer. Shield 310 is sized appropriately for the wearer's face in accordance with he dimensions described above. Goggles 300A also have stiffened area 311 similar to area 111A except shaped to create air bubble 312 when worn over the wearer's eyes; adhesive perimeter 320 with adhesive 97, similar to adhesive perimeter 120 with adhesive 99; and headlamp 250. Stiffened area 311 can be as stiff as the plastic used in conventional goggles, thereby protecting the wearer's eyes from, e.g., construction debris, To put on goggles 300A, the wearer presses adhesive 97 in perimeter area 320 to her/his face. To remove goggles 300A, the wearer gently separates adhesive 97 from her/his face.

Goggles 300B are similar to goggles 300A, and also include forehead air filter cartridge 36F that removably inserts into opening 37F, and headband 380 similar to headband 180, discussed above. Air filter cartridge 36F functions to transmit heat and moisture from air bubble 312 to the environment outside goggles 300B, and to protect the wearer's eyes from airborne pathogens.

In some embodiments, goggles 300 have glasses gates as described above.

Advantages of goggles 300 compared to conventional plastic goggles include, but are not limited to:
1. Comfort. The adhesive perimeter of goggles 300, in combination with its light weight and flexibility, eliminates the need for ear loops or behind-the-head ties, thereby eliminating chafing or discomfort from such ear loops or ties.
2. Health-promotion. Because the adhesive in the perimeter of goggles 300 is used for skin protection and healing, the wearer's skin may be improved by extended wearing of goggles 300.
3. Protection for the wearer against airborne pathogens. Goggles 300 serves as an air barrier, protecting the wearer's eyes from airborne pathogens.
4. Easy to apply. No particular skill or training is required to apply goggles 300. Goggles 300 can be applied kinesthetically, by simply running one's finger around the perimeter to ensure an entire seal. Goggles 300 can be applied visually, by the wearer inspecting their appearance in a mirror. A third-party can apply goggles 300 to the wearer.
5. Easy to remove. No particular skill or training is required to remove goggles 300. Either the wearer or a third party gently breaks the perimeter seal at one or more points, then gently lifts the rest of the perimeter from the wearer's face.
6. Reusable. Goggles 300 are resilient, reusable, and recyclable.
7. Easy to clean. No special equipment or preparations are needed to clean goggles 300; they can be washed or wiped with soap and water, or sterilized in a microwave or autoclave.
8. Durable. Because shield 310 is impervious to water, splashing or spitting from a third party does not harm goggles 300, and the wearer does not need to immediately change to a new mask but instead can continue with their activity.
9. Appropriate protection. Area 311 of goggles 300A is stiffer than area 111A of mask 100A, because the wearer's eyes are more susceptible to explosive force and to small edged projectiles, such as construction chips, than the wearer's lower face. Flexibility is less important for goggles 300A than for mask 100A, because the lower face has jaw mobility and large muscle movements for smiles, whereas the upper face is capable of only smaller muscle movements such as winking.

Although illustrative embodiments of the present invention, and various modifications thereof, have been described in detail herein with reference to the accompanying drawings, it is to be understood that the invention is not limited to this precise embodiment and the described modifications, and that various changes and further modifications may be effected therein by one skilled in the art without departing from the scope or spirit of the invention as defined in the appended claims

What is claimed is:

1. A face mask for a human wearer, comprising:
a shield for covering a portion of a face of the wearer, the shield being formed of a material that blocks airborne pathogens, the shield having a border area on one side of the shield that extends around a pen of the shield, and
a hydrogel layer covering the border area of the shield, for removably adhering to the wearer's face via hydrogel adhesion, so that the face mask can be worn without a structure for applying force to keep the face mask on the wearer's face.

2. The face mask of claim 1, wherein the material of the shield is clear flexible silicone.

3. The face mask of claim 1, wherein the shield covers a lower portion of the wearer's face including the wearer's mouth and nostril openings.

4. The face mask of claim 3, wherein the shield includes at least one opening, and further comprising at least one removable air filter cartridge that inserts into and removes from the opening in the shield, the air filter cartridge for reducing moisture and heat from the inside of the mask when the mask is affixed to the wearer's face by the hydrogel layer, and for enabling the wearer to safely inhale and exhale in an environment containing airborne pathogens.

5. The face mask of claim 4, wherein the air filter cartridge includes an air filter, an activated carbon filter, and a frame for holding the air filter and the activated carbon filter.

6. The face mask of claim 3, further comprising a breathing valve that opens during exhalation and closes during inhalation.

7. The face mask of claim 3, further comprising a headband for keeping the mask on the wearer's body even when the mask is not affixed to the wearer's face by the hydrogel layer.

8. The face mask of claim 1, wherein the shield covers an upper portion of the wearer's face including the wearer's eyes.

9. The face mask of claim 8, wherein the shield includes at least one opening, and further comprising at least one removable air filter cartridge that inserts into and removes from the opening in the shield, the air filter cartridge for reducing moisture and heat from the inside of the mask when the mask is affixed to the wearer's face by the hydrogel layer or non-toxic emulsion adhesive layer.

10. The face mask of claim 9, wherein the air filter cartridge includes an air filter, an activated carbon filter, and a frame for holding the air filter and the activated carbon filter.

11. The face mask of claim 8, further comprising a headlamp for illuminating what the wearer is looking at.

12. The face mask of claim 8, further comprising a glasses gate for enabling arms of glasses worn by the wearer to extend through the mask and over the wearer's ears.

13. The face mask of claim 8, further comprising a headband for keeping the mask on the wearer's body even when the mask is not affixed to the wearer's face by the hydrogel layer.

14. The face mask of claim 1, wherein the shield covers a majority of the wearer's face.

15. The face mask of claim 14, wherein the shield includes at least one opening, and further comprising at least one removable air filter cartridge that inserts into and removes from the opening in the shield, the air filter cartridge for reducing moisture and heat from the inside of the mask when the mask is affixed to the wearer's face by the hydrogel layer, and for enabling the wearer to safely inhale and exhale in an environment containing airborne pathogens.

16. The face mask of claim 15, wherein the air filter cartridge includes an air filter, an activated carbon filter, and a frame for holding the air filter and the activated carbon filter.

17. The face mask of claim 14, further co sing a breathing valve that opens during exhalation and closes during inhalation.

18. The face mask of claim 14, further comprising a headlamp for illuminating what the wearer is looking at.

19. The face mask of claim 14, further sing a glasses gate for enabling arms of glasses worn by the wearer to extend through the mask and over the wearer's ears.

20. The face mask of claim 14, further comprising a headband for keeping the mask on the wearer's body even when the mask is not affixed to the wearer's face by the hydrogel layer.
